(19) United States
(12) Patent Application Publication
(10) Pub. No.: US 2026/0188097 A1
(43) Pub. Date: Jul. 2, 2026

(54) Patient Abnormality Observation During Medical Imaging

(71) Applicant: Siemens Healthineers AG, Forchheim (DE)

(72) Inventors: Jia Min Liang, Shenzhen (CN); Fang Yong Sun, Shenzhen (CN); Jun Xiong, Shenzhen (CN); Xue Feng Zhao, Shenzhen (CN); Jing Guo, Shenzhen (CN)

(73) Assignee: Siemens Healthineers AG, Forchheim (DE)

(21) Appl. No.: 19/550,490

(22) Filed: Feb. 26, 2026

(30) Foreign Application Priority Data
Feb. 26, 2005 (CN) 202510227750.0

(51) Int. Cl. G08B 21/04

(52) U.S. Cl. CPC G08B 21/0476; G08B 21/0407

(57) ABSTRACT

A method for observing patient abnormality during medical imaging, including: acquiring behavioral expression monitoring data of a patient during medical imaging; based on the behavioral expression monitoring data, judging whether human sound-making behavior is present and/or whether bodily movement behavior is present; and at least based on whether human sound-making behavior is present and/or whether bodily movement behavior is present, judging whether the patient exhibits abnormal behavior; and upon determining that the patient exhibits abnormal behavior, generating a signal to notify an abnormality observer. The method for observing patient abnormality allows the condition of a patient during medical imaging to be observed more conveniently. In addition, a system for observing patient abnormality is provided to realize the method for observing patient abnormality.

### TECHNICAL FIELD

The present disclosure relates to the field of medical imaging, in particular to a method for observing patient abnormality during medical imaging and a system for observing patient abnormality.

### BACKGROUND ART

During medical imaging, the technician must monitor the condition of the patient in real time to prevent accidents. At present, a squeeze ball can be provided to the patient in advance; if the patient feels abnormal during medical imaging, he can trigger an alert to notify the technician by operating the squeeze ball. However, many difficulties are associated with using a squeeze ball, for example, but not limited to the following: erroneous operation by the patient will affect the procedure; the squeeze ball may become jammed in the equipment; a patient with a hand injury will find it difficult to use; and so on.

### SUMMARY

An objective of the present disclosure is to provide a method for observing patient abnormality during medical imaging, which allows the condition of a patient during medical imaging to be observed more conveniently.

Another objective of the present disclosure is to provide a system for observing patient abnormality, which allows the condition of a patient during medical imaging to be observed more conveniently.

The present disclosure provides a method for observing patient abnormality during medical imaging, comprising: S10: acquiring behavioral expression monitoring data of a patient during medical imaging; S20: based on the behavioral expression monitoring data, judging whether human sound-making behavior is present and/or whether bodily movement behavior is present; and at least based on whether human sound-making behavior is present and/or whether bodily movement behavior is present, judging whether the patient exhibits abnormal behavior; and S30: upon determining that the patient exhibits abnormal behavior, generating a signal to notify an abnormality observer.

In the method for observing patient abnormality during medical imaging, a judgment is made regarding whether the patient exhibits abnormal behavior based on the behavioral expression monitoring data of the patient; there is no need for the patient to operate a squeeze ball, so the condition of the patient during medical imaging can be monitored more conveniently.

In another schematic aspect of the method for observing patient abnormality during medical imaging, the behavioral expression monitoring data comprises audio data and/or video data. The audio data records the sound of a space in which the patient should be located during medical imaging. The video data records images of the space in which the patient should be located during medical imaging. This facilitates the acquisition of behavioral expression monitoring data. S20 comprises: S21: judging whether human sound-making behavior is present based on the audio data; and/or S22: judging whether bodily movement behavior is present based on the video data.

In another schematic aspect of the method for observing patient abnormality during medical imaging, S20 further comprises:
determining that the patient exhibits abnormal behavior if condition 1 is met: human sound-making behavior and bodily movement behavior are simultaneously present, the extent of movement is greater than a first extent threshold, and the speed of movement is greater than a first speed threshold;
determining that the patient exhibits abnormal behavior if condition 2 is met: human sound-making behavior and bodily movement behavior are simultaneously present, and the extent of movement is greater than a second extent threshold, wherein the second extent threshold is greater than the first extent threshold;
determining that the patient exhibits abnormal behavior if condition 3 is met: human sound-making behavior and bodily movement behavior are simultaneously present, and the speed of movement is greater than a second speed threshold, wherein the second speed threshold is greater than the first speed threshold;
determining that the patient exhibits abnormal behavior if condition 4 is met: bodily movement behavior is present, and an action is a specific action; or
if human sound-making behavior and/or bodily movement behavior is present, and none of conditions 1, 2, 3, and 4 is met, then:
broadcasting speech to prompt the patient to perform a specific action if an abnormal situation occurs, then judging whether the patient has performed a specific action based on the video data of a preset time period following broadcast of the speech prompt, and if the judgment result is positive, determining that the patient exhibits abnormal behavior; and/or
displaying, on a display device, video content of the video data when bodily movement behavior is present and/or speech content obtained by recognition based on the audio data when human sound-making behavior is present, for viewing by the abnormality observer. This helps to judge more accurately whether the patient exhibits abnormal behavior.

In another schematic aspect of the method for observing patient abnormality during medical imaging, in S20, if human sound-making behavior and/or bodily movement behavior is present, the patient is determined as exhibiting abnormal behavior.

In another schematic aspect of the method for observing patient abnormality during medical imaging, in S20, if speech is externally broadcast during medical imaging, then a judgment is made regarding whether bodily movement behavior is present based on the video data; if no speech is externally broadcast during medical imaging, then a judgment is made regarding whether human sound-making behavior is present based on the audio data first, and upon determining that no human sound-making behavior is present based on the audio data, a judgment is then made regarding whether bodily movement behavior is present based on the video data. This helps to prevent broadcast speech from influencing judgment results.

In another schematic aspect of the method for observing patient abnormality during medical imaging, S21 comprises: S211: subjecting the audio data to preprocessing, the preprocessing comprising filtering and/or frequency domain conversion, the filtering being used to filter out a portion of the audio data that is greater than a frequency threshold, the frequency threshold being in the range of 300 Hz-1000 Hz, and the frequency domain conversion being used to convert the audio data to frequency domain data; and S212: judging whether human sound-making behavior is present based on data resulting from the preprocessing. This helps to improve algorithm robustness. Frequency domain conversion helps to achieve precise extraction of speech characteristics and effective noise removal.

In another schematic aspect of the method for observing patient abnormality during medical imaging, if speech is externally broadcast during medical imaging, the preprocessing further comprises filtering out a portion of the audio data that corresponds to the externally broadcast speech.

In another schematic aspect of the method for observing patient abnormality during medical imaging, in S21, a judgment is made regarding whether human sound-making behavior is present based on the audio data using a classification neural network. This helps to improve judgment accuracy.

In another schematic aspect of the method for observing patient abnormality during medical imaging, in S30, upon determining that human sound-making behavior is present and determining that the patient exhibits abnormal behavior, speech recognition is performed based on the audio data, so as to obtain speech content data, and a signal is generated according to the speech content data to notify the abnormality observer of content of the patient's talk. This makes it easier for the abnormality observer to understand the content of the patient's talk.

In another schematic aspect of the method for observing patient abnormality during medical imaging, S22 comprises: S221: based on the video data, judging whether a person is present in the space in which the patient should be located; and S222: if it is determined that a person is present in the space in which the patient should be located, judging whether bodily movement behavior is present based on the video data. This helps to improve judgment accuracy.

In another schematic aspect of the method for observing patient abnormality during medical imaging, S221 comprises: S2211: extracting a video stream from the video data, using a first frame of the video stream as a region reference image, using a target detection algorithm based on a convolutional neural network to predict a center position, a length and a width of a target region box of a human body in the region reference image, so as to determine a target region box, and issuing a confidence score, the confidence score indicating the degree of certainty of the target detection algorithm that an object in the target region box is a person; and S2212: comparing the confidence score with a preset confidence threshold, and if the confidence score is greater than the preset confidence threshold, determining that a person is present in the space in which the patient should be located. This helps to improve judgment accuracy.

In another schematic aspect of the method for observing patient abnormality during medical imaging, S222 comprises: S2221: cutting out a portion of each frame of the video stream that lies within the target region box; S2222: using a key point detection model to predict a key point position of a human body in the cut-out portion of each frame; and S2223: using a predictive model to judge whether bodily movement behavior is present based on the key point position of the human body in the cut-out portion of each frame. This helps to improve judgment accuracy.

In another schematic aspect of the method for observing patient abnormality during medical imaging, the generated signal for notifying the abnormality observer is configured to be able to control a sound-emitting device to emit a prompt sound, a display device to display prompt information, and/or an indicator lamp to switch between bright and dark states. This makes it easier for the abnormality observer to receive the signal.

The present disclosure further provides a system for observing patient abnormality, comprising a memory. The memory stores a computer program. When executed by a processor, the computer program can realize the method for observing patient abnormality described above. In the system for observing patient abnormality, a judgment can be made regarding whether the patient exhibits abnormal behavior based on the behavioral expression monitoring data of the patient; there is no need for the patient to operate a squeeze ball, so the condition of the patient during medical imaging can be monitored more conveniently.

In another schematic aspect of the system for observing patient abnormality, the system for observing patient abnormality further comprises a sound acquisition device and/or an image acquisition device. The sound acquisition device is used to acquire the sound of a space in which a patient should be located during medical imaging, so as to generate audio data as behavioral expression monitoring data. The image acquisition device is used to acquire images of the space in which the patient should be located during medical imaging, so as to generate video data as behavioral expression monitoring data. This facilitates the acquisition of behavioral expression monitoring data.

### BRIEF DESCRIPTION OF THE DRAWINGS

The accompanying drawings below merely illustrate and explain the present disclosure schematically, without limiting the scope thereof.

FIG. 1 is a flowchart of a schematic method for observing patient abnormality during medical imaging.

FIGS. 2-4 illustrate three specific examples of the method for observing patient abnormality.

FIG. 5 is a flowchart of the specific steps of S221.

FIG. 6 is a flowchart of the specific steps of S222.

FIG. 7 is a structural schematic drawing intended to illustrate a schematic aspect of a system for observing patient abnormality.

### KEY TO LABELS

10 memory
30 sound acquisition device
50 image acquisition device
60 space in which the patient should be located

### DETAILED DESCRIPTION

To enable a clearer understanding of the technical features, objectives, and effects of the disclosure, particular aspects of the present disclosure are now explained with reference to the accompanying drawings, in which identical labels indicate structurally identical components or components with similar structures but identical functions.

As used herein, “schematic” means “serving as an instance, example, or illustration”. No drawing or aspect described herein as “schematic” should be interpreted as a more preferred or more advantageous technical solution.

For clarity of the drawings, only those parts relevant to the present disclosure are shown schematically in the drawings; they do not represent the actual structure thereof as a product.

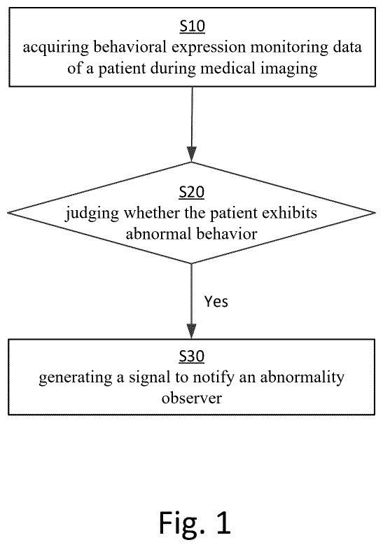

FIG. 1 is a flowchart of a schematic aspect of a method for observing patient abnormality during medical imaging. The medical imaging is, for example, but without limitation, X-ray imaging, computed tomography, magnetic resonance imaging, ultrasound imaging, nuclear medical imaging, etc. As shown in FIG. 1, the method for observing patient abnormality during medical imaging comprises S10 to S30 below.

S10: acquiring behavioral expression monitoring data of a patient during medical imaging. Behavioral expression monitoring data is data related to the behavioral expression of the patient. Behavioral expression is a process whereby an individual conveys its own inner psychological state and information, such as thinking, emotions, intentions, and attitudes, through various types of behavior; it is an important component part of human communication and interaction, involving many aspects such as bodily actions and sound characteristics. In this schematic aspect, the behavioral expression monitoring data, for example, comprises audio data and/or video data. The audio data records the sound of a space in which the patient should be located during medical imaging. The video data records images of the space in which the patient should be located during medical imaging. This facilitates the acquisition of behavioral expression monitoring data, but there is no limitation to this.

As used herein, the expression “space in which the patient should be located”means the largest spatial range used for positioning the patient during medical imaging. To meet imaging requirements, the patient cannot go outside this largest spatial range during medical imaging.

The audio data is, for example, acquired from a sound acquisition device. The sound acquisition device is, for example, a microphone. The sound acquisition device is used to acquire sound of the space in which the patient should be located during medical imaging, so as to generate audio data. The video data is, for example, acquired from an image acquisition device. The image acquisition device is, for example, a camera. The image acquisition device is used to acquire images of the space in which the patient should be located during medical imaging, so as to generate video data.

S20: Based on the behavioral expression monitoring data, judging whether human sound-making behavior is present and/or whether bodily movement behavior is present; and at least based on whether human sound-making behavior is present and/or whether bodily movement behavior is present, judging whether the patient exhibits abnormal behavior. In this schematic aspect, for example, in S20, if human sound-making behavior is present and/or bodily movement behavior is present, the patient is determined as exhibiting abnormal behavior.

Specifically, corresponding to audio data as behavioral expression monitoring data, S20 comprises S21: based on audio data, judging whether human sound-making behavior is present. This judgment is realized by artificial intelligence technology, for example.

Corresponding to video data as behavioral expression monitoring data, S20 comprises S22: based on video data, judging whether bodily movement behavior is present. This judgment is realized by artificial intelligence technology, for example.

In some schematic aspects, the behavioral expression monitoring data comprises both audio data and video data. We can, for example, set rules for using these two types of data. For example, in S20, if speech is externally broadcast during medical imaging, then a judgment is made regarding whether bodily movement behavior is present based on video data. The expression “speech is externally broadcast during medical imaging,” for example, means that: speech emitted by an additional person, who is not in the space in which the patient should be located, is broadcast during medical imaging. A non-limiting example is a prompt speech that is broadcast via a loudspeaker and used to guide the patient. Since the broadcast speech causes interference, judging whether the patient exhibits abnormal behavior based on video data is more accurate. If no speech is externally broadcast during medical imaging, then a judgment can be made regarding whether human sound-making behavior is present based on audio data first, and upon determining that no human sound-making behavior is present based on audio data, a judgment can then be made regarding whether bodily movement behavior is present based on video data. This helps to prevent broadcast speech from influencing judgment results.

Of course, in other schematic aspects, the following is also possible: if no speech is externally broadcast during medical imaging, then a judgment is made regarding whether bodily movement behavior is present based on video data first, and upon determining that no bodily movement behavior is present based on video data, a judgment is made regarding whether human sound-making behavior is present based on audio data. In other schematic aspects, the following is also possible: if no speech is externally broadcast during medical imaging, a judgment is made regarding whether bodily movement behavior is present based on video data, and at the same time, a judgment is made regarding whether human sound-making behavior is present based on audio data.

In other schematic aspects, the following is also possible: if speech is externally broadcast during medical imaging, a judgment is made regarding whether bodily movement behavior is present based on video data, and at the same time, a judgment is made regarding whether human sound-making behavior is present based on audio data. In this case, for example, a portion of the audio data that corresponds to the externally broadcast speech can be filtered out prior to judgment, so as to improve judgment accuracy.

Specifically, in a schematic aspect, S21, for example, comprises S211 and S212 below.

S211: subjecting audio data to preprocessing, the preprocessing comprising filtering and/or frequency domain conversion. Filtering is used to filter out a portion of the audio data that is greater than a frequency threshold, wherein the frequency threshold is in the range of 300 Hz-1000 Hz; further, the frequency threshold is in the range of 500 Hz-1000 Hz; even further, the frequency threshold is 1000 Hz. Since the range of fundamental frequency of the human voice is mainly below 1 kHz, this helps to improve algorithm robustness. Filtering is realized by means of a low-pass filter, for example. Frequency domain conversion is used to convert the audio data to frequency domain data, so as to achieve precise extraction of speech characteristics and effective noise removal. In a schematic aspect, if speech is externally broadcast during medical imaging, the preprocessing, for example, further comprises filtering out a portion of the audio data that corresponds to the externally broadcast speech.

S212: based on data resulting from preprocessing, judging whether human sound-making behavior is present using artificial intelligence technology.

Of course, in other schematic aspects, the audio data need not be preprocessed; instead, a judgment can be made regarding whether human sound-making behavior is present based on the audio data directly, using artificial intelligence technology.

Specifically, in a schematic aspect, in S21, a judgment is made regarding whether human sound-making behavior is present based on the audio data, for example, using a classification neural network. This helps to improve judgment accuracy, but there is no limitation to this. The classification neural network is, for example, a fully connected layer classification neural network. A fully connected layer classification neural network C comprises an input layer, a hidden layer, and an output layer. The number of nodes in the input layer is the length of the input audio data (corresponding to the case where no preprocessing is performed), or data resulting from preprocessing of the audio data in S211, and the number of nodes in the output layer is 1. The output layer outputs a probability y_{pred}=C(X) that human sound-making behavior is present, wherein X is the input audio data or data resulting from preprocessing of the audio data in S211. Regarding the hidden layer, different numbers of layers and different numbers of nodes may, for example, be set according to the data complexity.

During medical imaging, when it is necessary to judge whether human sound-making behavior is present based on audio data, the audio data (corresponding to the case where no preprocessing is performed), or data resulting from preprocessing of the audio data in S211, X (X0, X1, X2, X3, . . . , Xn) is inputted into the fully connected layer classification neural network, to obtain a classification result y_{pred} for whether human sound-making behavior is present. When training the fully connected layer classification neural network, an L1 distance loss function Loss_{L1}=∥y−y_{pred}∥1 for example, is used, wherein y is the true classification label. If the audio data comprises the human voice, then y is 1, otherwise y is 0.

In other schematic aspects, a convolutional neural network or other deep learning or machine learning method may also be used to train a classifier for whether human sound-making behavior is present, to enable judgment of whether human sound-making behavior is present based on audio data.

In a schematic aspect, S22 for example comprises S221 and S222 below.

S221: based on video data, judging whether a person is present in the space in which the patient should be located using artificial intelligence technology.

S222: if it is determined that a person is present in the space in which the patient should be located, judging whether bodily movement behavior is present based on the video data using artificial intelligence technology. This helps to improve judgment accuracy.

S30: upon determining that the patient exhibits abnormal behavior, generating a signal to notify an abnormality observer, who is for example a technician or another person responsible for monitoring the patient. Specifically, the generated signal for notifying the abnormality observer is, for example, configured to be able to control a sound-emitting device to emit a prompt sound, a display device to display prompt information, and/or an indicator lamp to switch between bright and dark states. This makes it easier for the abnormality observer to receive the signal. The prompt sound emitted by the sound-emitting device is, for example, speech and/or a prompt sound such as “beep beep beep” or “deedeedee”; the prompt information displayed by the display device is, for example, text, a pattern, and/or an image generated according to the video data but is not limited to this.

In a schematic aspect, in S30: upon determining that human sound-making behavior is present and determining that the patient exhibits abnormal behavior, speech recognition is for example performed based on the audio data using artificial intelligence technology, so as to obtain speech content data, and a signal is generated according to the speech content data to notify the abnormality observer of the content of the patient's talk. This makes it easier for the abnormality observer to understand the content of the patient's talk.

The method for observing patient abnormality described above may, for example, be repeated as required during medical imaging.

In the method for observing patient abnormality during medical imaging, a judgment is made regarding whether the patient exhibits abnormal behavior based on the behavioral expression monitoring data of the patient; there is no need for the patient to operate a squeeze ball, so the condition of the patient during medical imaging can be monitored more conveniently.

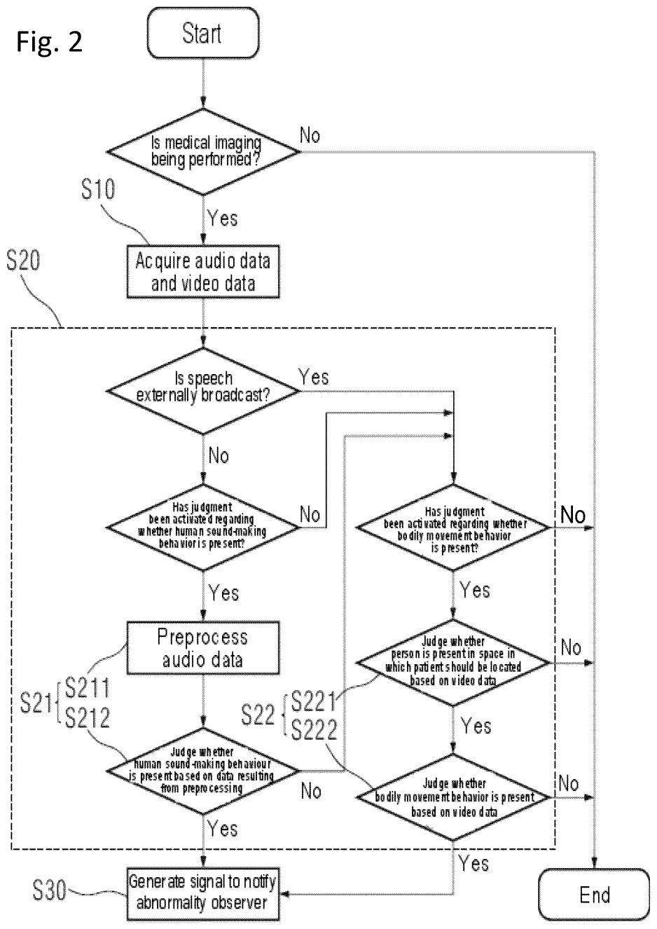

FIGS. 2-4 respectively illustrate three specific examples of the method for observing patient abnormality shown in FIG. 1.

In the method for observing patient abnormality shown in FIG. 2, the behavioral expression monitoring data comprises both audio data and video data. After the procedure begins, a judgment is first made regarding whether medical imaging is being performed; if the judgment result is positive, then the method proceeds to S10, and if the judgment result is negative, the method ends. In S20, if it is determined that no speech is externally broadcast and that judgment has been activated regarding whether human sound-making behavior is present, the method proceeds to S21. If it is determined that speech is externally broadcast and that judgment has been activated regarding whether bodily movement behavior is present, the method proceeds to S22. If it is determined that no speech is externally broadcast, that judgment has not been activated regarding whether human sound-making behavior is present, and that judgment has been activated regarding whether bodily movement behavior is present, the method proceeds to S22. If the judgment result of S212 is negative and judgment has been activated regarding whether bodily movement behavior is present, the method proceeds to S22. If the judgment result of S212 or S222 is positive, the method proceeds to S30.

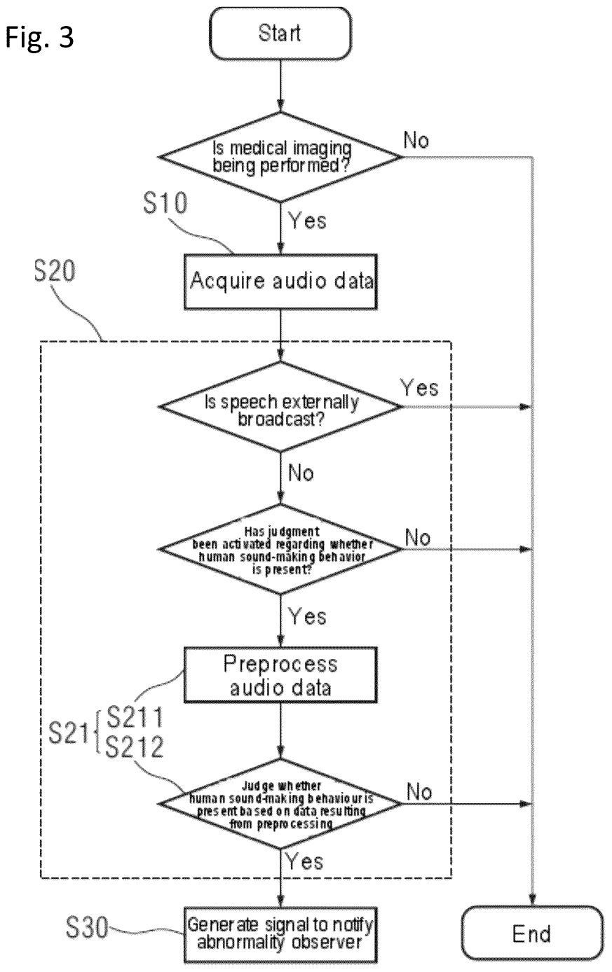

In the method for observing patient abnormality shown in FIG. 3, the behavioral expression monitoring data only comprises audio data. After the procedure begins, a judgment is first made regarding whether medical imaging is being performed; if the judgment result is positive, then the method proceeds to S10, and if the judgment result is negative, the method ends. In S20, if it is determined that no speech is externally broadcast and that judgment has been activated regarding whether human sound-making behavior is present, the method proceeds to S21. If the judgment result of S212 is positive, the method proceeds to S30.

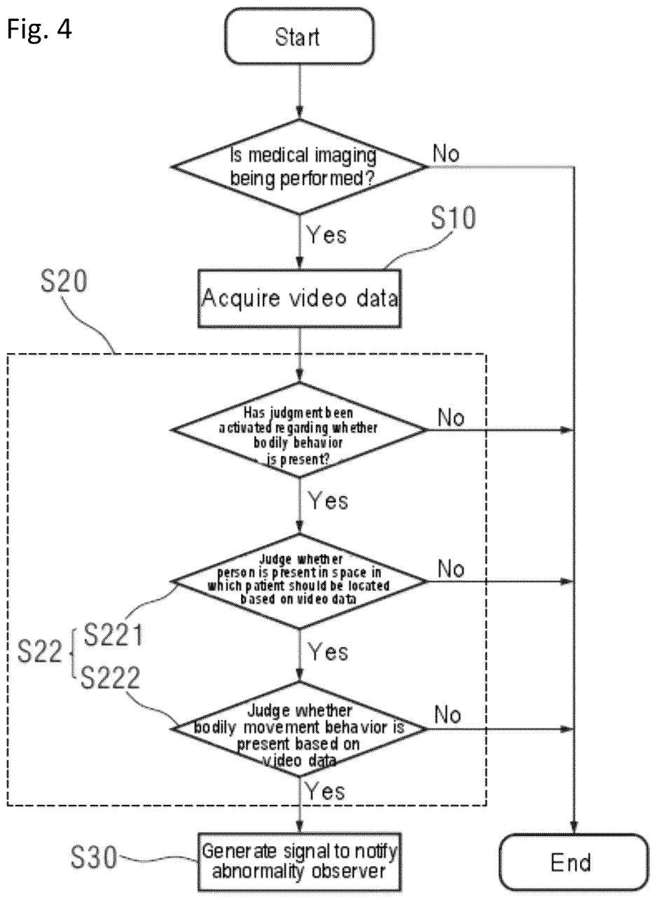

In the method for observing patient abnormality shown in FIG. 4, the behavioral expression monitoring data only comprises video data. After the procedure begins, a judgment is first made regarding whether medical imaging is being performed; if the judgment result is positive, then the method proceeds to S10, and if the judgment result is negative, the method ends. In S20, if it is determined that judgment has been activated regarding whether bodily movement behavior is present, the method proceeds to S22. If the judgment result of S222 is positive, the method proceeds to S30.

In the three specific examples illustrated in FIGS. 2-4 , with regard to “judging whether the patient exhibits abnormal behavior based on whether human sound-making behavior is present and/or whether bodily movement behavior is present” in S20, the specific solution adopted is: if human sound-making behavior and/or bodily movement behavior is present, the patient is determined as exhibiting abnormal behavior. Thus, to save on programming, if the judgment result of S212 or S222 is positive, the method proceeds directly to S30.

It will be understood that, based on the specific examples above, if the judgment of the system regarding whether human sound-making behavior is present is defined to be always in an activated state, there is no need to perform the step of judging whether judgment has been activated regarding whether human sound-making behavior is present. By the same principle, if the judgment of the system regarding whether bodily movement behavior is present is defined to be always in an activated state, there is no need to perform the step of judging whether judgment has been activated regarding whether bodily movement behavior is present.

In the schematic aspects above, with regard to “judging whether the patient exhibits abnormal behavior based on whether human sound-making behavior is present and/or whether bodily movement behavior is present” in S20, the solution adopted is: if human sound-making behavior and/or bodily movement behavior is present, the patient is determined as exhibiting abnormal behavior. However, there is no restriction on this. In other schematic aspects, the solution may comprise any one of the following, or a combination of any of the following:
determining that the patient exhibits abnormal behavior if condition 1 is met: human sound-making behavior and bodily movement behavior are simultaneously present, the extent of movement is greater than a first extent threshold, and the speed of movement is greater than a first speed threshold;
determining that the patient exhibits abnormal behavior if condition 2 is met: human sound-making behavior and bodily movement behavior are simultaneously present, and the extent of movement is greater than a second extent threshold, wherein the second extent threshold is greater than the first extent threshold;
determining that the patient exhibits abnormal behavior if condition 3 is met: human sound-making behavior and bodily movement behavior are simultaneously present, and the speed of movement is greater than a second speed threshold, wherein the second speed threshold is greater than the first speed threshold;
determining that the patient exhibits abnormal behavior if condition 4 is met: bodily movement behavior is present, and an action is a specific action (for example, but not limited to lifting the leg or hand, etc.); or
if human sound-making behavior and/or bodily movement behavior is present, and none of conditions 1, 2, 3, and 4 is met, then:
broadcasting speech (for example, externally broadcast speech or earphone broadcast speech) to prompt the patient to perform a specific action (for example but not limited to lifting the leg or hand, etc.) if an abnormal situation occurs, then judging whether the patient has performed a specific action based on video data of a preset time period following broadcast of the speech prompt, and if the judgment result is positive, determining that the patient exhibits abnormal behavior;
displaying, on a display device, video content of the video data when bodily movement behavior is present and/or speech content obtained by recognition based on the audio data when human sound-making behavior is present, for viewing by the abnormality observer. After viewing the content, the abnormality observer makes a human judgment regarding whether the patient exhibits abnormal behavior. This helps to judge more accurately whether the patient exhibits abnormal behavior.

In the solution above, the first extent threshold, the first speed threshold, the second extent threshold, the second speed threshold, and the specific action may, for example, be set according to requirements.

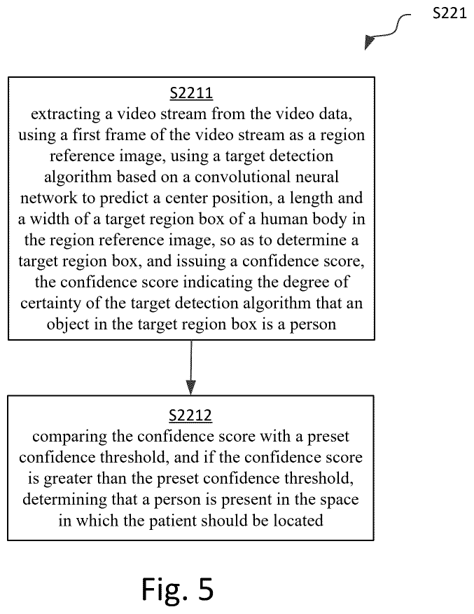

As shown in FIG. 5, in a schematic aspect, S221, for example, comprises S2211 and S2212 below.

S2211: extracting a video stream from video data, using a first frame of the video stream as a region reference image, using a target detection algorithm based on a convolutional neural network to predict a center position, a length and a width of a target region box of a human body in the region reference image, so as to determine a target region box, and issuing a confidence score. The confidence score indicates the degree of certainty of the target detection algorithm that an object in the target region box is a person.

Specifically, the region reference image is used as an input, a convolutional neural network is used to extract image potential characteristics, then a fully connected layer classification neural network is used to obtain multiple sets of possible parameters to be predicted by a regression task, and a confidence score for each set of parameters. In a training stage, the parameters of the convolutional neural network and the fully connected layer classification neural network are updated by gradient backpropagation of the loss values obtained by calculation of the loss function. The loss value calculates the difference of the absolute distance L1 or the square root distance L2 between a predicted value and a true value. During use, the set of values with the highest confidence score is screened out as the final result. In a schematic aspect, the number of layers in the convolutional neural network and the fully connected layer classification neural network can be set to different values according to the actual data complexity.

S2212: comparing the confidence score with a preset confidence threshold, and if the confidence score is greater than the preset confidence threshold, determining that a person is present in the space in which the patient should be located. This helps to improve judgment accuracy.

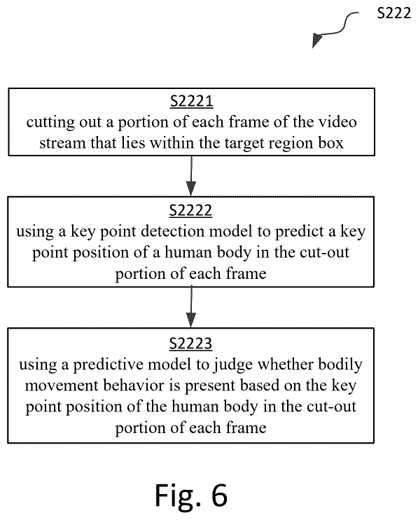

As shown in FIG. 6, in a schematic aspect, S222, for example, comprises S2221 to S2223 below.

S2221: cutting out a portion of each frame of the video stream that lies within the target region box.

S2222: using a key point detection model to predict key point positions of a human body in the cut-out portion of each frame. The key point detection model, for example, consists of multiple convolution layers and deconvolution layers. The key points of the human body are, for example, located at the head and the four limbs. During medical imaging, e.g., CT or MRI, the head and the four limbs are the parts of the patient that are most likely to perform abnormal actions. Thus, this configuration helps to improve judgment accuracy. However, there is no restriction on this.

S2223: using a predictive model to judge whether bodily movement behavior is present based on the key point position of the human body in the cut-out portion of each frame. Specifically, the predictive model can, for example, judge that bodily movement behavior is present if a displacement of any key point is greater than a set threshold. The predictive model, for example, employs a fully connected layer classification neural network with multiple layers. This helps to improve judgment accuracy.

In other schematic aspects, the following is also possible: extracting a particular frame from video data, obtaining parameters of a target region box and a corresponding confidence score by the method described in S2211, and judging whether a person is present in the space in which the patient should be located by the method described in S2212; if it is determined that a person is present in the space in which the patient should be located, cutting out a portion of the frame that lies within the target region box, using a key point detection model to predict key point positions of a human body in the cut-out portion of the frame, and using a predictive model to judge whether bodily movement behavior is present based on the key point positions of the human body in the cut-out portion of the frame.

In other schematic aspects, it is also possible to judge whether bodily movement behavior is present through a series of procedures, such as constructing a suitable model and performing characteristic extraction and analysis, with multiple frames in video data as input data, by means of a deep learning method such as a convolutional neural network, and an abnormal action classification algorithm in machine learning.

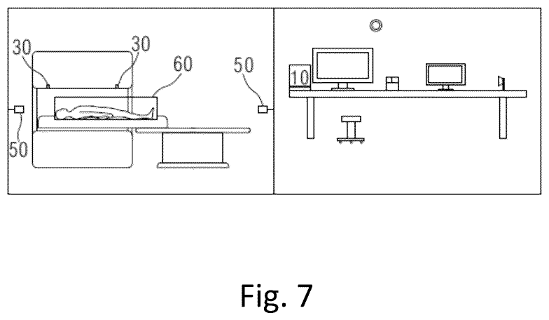

FIG. 7 is a structural schematic drawing intended to illustrate a schematic aspect of a system for observing patient abnormality. In this schematic aspect, magnetic resonance imaging is taken as an example for illustration. As shown in FIG. 7, the system for observing patient abnormality comprises a memory 10. The memory 10 stores a computer program. When executed by a processor, the computer program can realize the method for observing patient abnormality described above. In the system for observing patient abnormality, a judgment can be made regarding whether the patient exhibits abnormal behavior based on the behavioral expression monitoring data of the patient; there is no need for the patient to operate a squeeze ball, so the condition of the patient during medical imaging can be monitored more conveniently.

As shown in FIG. 7, in a schematic aspect, the system for observing patient abnormality further comprises a sound acquisition device 30 and an image acquisition device 50. The sound acquisition device 30 is used to acquire sound of a space 60 in which the patient should be located during medical imaging, so as to generate audio data as behavioral expression monitoring data. The image acquisition device 50 is used to acquire images of the space 60 in which the patient should be located during medical imaging, so as to generate video data as behavioral expression monitoring data. The sound acquisition device 30 is a microphone, for example, and the image acquisition device 50 is a camera, for example, but there is no limitation to this. This facilitates the acquisition of behavioral expression monitoring data.

It should be understood that although the description herein is based on various aspects, it is by no means the case that each aspect contains only one independent technical solution. Such a method of presentation is adopted herein purely for the sake of clarity. Those skilled in the art should consider the description in its entirety. The technical solutions in the various aspects could also be suitably combined to form other aspects understandable to those skilled in the art.

The series of detailed explanations set out above are merely particular explanations of feasible aspects of the present disclosure and are not intended to limit the scope of protection thereof. All equivalent aspects or changes made without departing from the artistic spirit of the present disclosure, such as combinations, divisions, or repetitions of features, shall be included in the scope of protection of the present disclosure.

1. A method for observing patient abnormality during medical imaging, comprising:
S10: acquiring behavioral expression monitoring data of a patient during medical imaging;
S20: based on the behavioral expression monitoring data, judging whether human sound-making behavior is present and/or whether bodily movement behavior is present; and at least based on whether human sound-making behavior is present and/or whether bodily movement behavior is present, judging whether the patient exhibits abnormal behavior; and
S30: upon determining that the patient exhibits abnormal behavior, generating a signal to notify an abnormality observer.

2. The method for observing patient abnormality during medical imaging as claimed in claim 1,
wherein the behavioral expression monitoring data comprises audio data and/or video data, the audio data recording sound of a space in which the patient should be located during medical imaging, and the video data recording images of the space in which the patient should be located during medical imaging, and
wherein S20 comprises:
S21: judging whether human sound-making behavior is present based on the audio data; and/or
S22: judging whether bodily movement behavior is present based on the video data.

3. The method for observing patient abnormality during medical imaging as claimed in claim 2, wherein S20 further comprises:
determining that the patient exhibits abnormal behavior if condition 1 is met: human sound-making behavior and bodily movement behavior are simultaneously present, an extent of movement is greater than a first extent threshold, and a speed of movement is greater than a first speed threshold;
determining that the patient exhibits abnormal behavior if condition 2 is met: human sound-making behavior and bodily movement behavior are simultaneously present, and the extent of movement is greater than a second extent threshold, wherein the second extent threshold is greater than the first extent threshold;
determining that the patient exhibits abnormal behavior if condition 3 is met: human sound-making behavior and bodily movement behavior are simultaneously present, and the speed of movement is greater than a second speed threshold, wherein the second speed threshold is greater than the first speed threshold;
determining that the patient exhibits abnormal behavior if condition 4 is met: bodily movement behavior is present, and an action is a specific action; or
if human sound-making behavior and/or bodily movement behavior is present, and none of conditions 1, 2, 3, and 4 is met, then:
broadcasting speech to prompt the patient to perform a specific action if an abnormal situation occurs, then judging whether the patient has performed a specific action based on the video data of a preset time period following broadcast of the speech prompt, and if the judgment result is positive, determining that the patient exhibits abnormal behavior; and/or
displaying, on a display device, video content of the video data when bodily movement behavior is present and/or speech content obtained by recognition based on the audio data when human sound-making behavior is present, for viewing by the abnormality observer.

4. The method for observing patient abnormality during medical imaging as claimed in claim 2, wherein S21 comprises:
S211: subjecting the audio data to preprocessing, the preprocessing comprising filtering and/or frequency domain conversion, the filtering being used to filter out a portion of the audio data that is greater than a frequency threshold, the frequency threshold being in a range of 300 Hz-2000 Hz, and the frequency domain conversion being used to convert the audio data to frequency domain data; and
S212: judging whether human sound-making behavior is present based on data resulting from the preprocessing.

5. The method for observing patient abnormality during medical imaging as claimed in claim 4, wherein if speech is externally broadcast during medical imaging, the preprocessing further comprises filtering out a portion of the audio data that corresponds to the externally broadcast speech.

6. The method for observing patient abnormality during medical imaging as claimed in claim 2, wherein in S30, upon determining that human sound-making behavior is present and determining that the patient exhibits abnormal behavior, speech recognition is performed based on the audio data, so as to obtain speech content data, and a signal is generated according to the speech content data to notify the abnormality observer of content of the patient's talk.

7. The method for observing patient abnormality during medical imaging as claimed in claim 2, wherein S22 comprises:
S221: based on the video data, judging whether a person is present in the space in which the patient should be located; and
S222: if it is determined that a person is present in the space in which the patient should be located, judging whether bodily movement behavior is present based on the video data.

8. The method for observing patient abnormality during medical imaging as claimed in claim 7, wherein S221 comprises:
S2211: extracting a video stream from the video data, using a first frame of the video stream as a region reference image, using a target detection algorithm based on a convolutional neural network to predict a center position, a length and a width of a target region box of a human body in the region reference image, so as to determine a target region box, and issuing a confidence score, the confidence score indicating a degree of certainty of the target detection algorithm that an object in the target region box is a person; and
S2212: comparing the confidence score with a preset confidence threshold, and if the confidence score is greater than the preset confidence threshold, determining that a person is present in the space in which the patient should be located.

9. The method for observing patient abnormality during medical imaging as claimed in claim 8, wherein S222 comprises:
S2221: cutting out a portion of each frame of the video stream that lies within the target region box;
S2222: using a key point detection model to predict a key point position of a human body in the cut-out portion of each frame; and
S2223: using a predictive model to judge whether bodily movement behavior is present based on the key point position of the human body in the cut-out portion of each frame.

10. The method for observing patient abnormality during medical imaging as claimed in claim 1, wherein the generated signal for notifying the abnormality observer is configured to be able to control a sound-emitting device to emit a prompt sound, a display device to display prompt information, and/or an indicator lamp to switch between bright and dark states.

11. A system for observing patient abnormality, wherein the system for observing patient abnormality comprises a non-transitory memory configured to store a computer program which, when executed by a processor, is configured to realize the method for observing patient abnormality as claimed in claim 1.

12. The system for observing patient abnormality as claimed in claim 11, wherein the system for observing patient abnormality further comprises:
a sound acquisition device configured to acquire sound of a space in which a patient should be located during medical imaging, so as to generate audio data as the behavioral expression monitoring data; and/or
an image acquisition device configured to acquire images of the space in which the patient should be located during medical imaging, so as to generate video data as the behavioral expression monitoring data.
